(19) United States
(12) Patent Application Publication
(10) Pub. No.: US 2022/0370683 A1
(43) Pub. Date: Nov. 24, 2022

(54) METHODS AND COMPOSITION TO TREAT TENDON INJURY

(71) Applicant: CORNELL UNIVERSITY, Ithaca, NY (US)

(72) Inventor: Nelly Andarawis-Puri, Ithaca, NY (US)

(21) Appl. No.: 17/751,399

(22) Filed: May 23, 2022

Related U.S. Application Data
(60) Provisional application No. 63/191,357, filed on May 21, 2021.

(51) Int. Cl. A61L 27/36; A61L 27/54; A61L 27/52; A61P 19/04

(52) U.S. Cl. CPC A61L 27/3633; A61L 27/3662; A61L 27/54; A61L 27/52; A61P 19/04; A61L 2300/414; A61L 2430/10

(57) ABSTRACT

Provided are compositions and methods relating to methods and compositions to treat tendon injury.

### CROSS-REFERENCE TO RELATED APPLICATIONS

This application claims the benefit of and priority to co-pending U.S. Provisional Patent Application No. 63/191,357, filed on May 21, 2021 entitled “METHODS AND COMPOSITION TO TREAT TENDON INJURY,” the contents of which is incorporated by reference herein in its entirety.

### STATEMENT REGARDING FEDERALLY SPONSORED RESEARCH

This invention was made with government support under Grant No. AR068301 awarded by the National Institutes of Health. The government has certain rights in the invention.

### TECHNICAL FIELD

The subject matter disclosed herein is generally directed to methods and compositions for treatment of tendon injury, and more particularly, biologic and hybrid materials and methods their use in treating tendon injury.

### BACKGROUND

The pathogenesis of tendinopathy spans multiple biologically different injury states. Tendinopathies account for over 30% of all musculoskeletal consultations. Acute tendon injuries result from macrotrauma but chronic injuries result from repetitive overuse. Despite this distinction, most seemingly acute injuries occur in a tendon weakened by accumulated fatigue damage. Accordingly, tendinopathy clinically presents with varied characteristics, depending on patient specific state of matrix degeneration, pain, and loss of function.

Conventional therapeutics for tendinopathies have led to mixed outcomes because the biological environment varies between different types and stages of tendon injuries. Distinctly different biological environments are associated with different states of tendon injuries. For instance, the anatomic location and local environment (i.e., intrasynovial versus extrasynovial) has been shown to affect the healing potential of torn and pathologic tendons. Even within the context of one tendon, the chronicity of the injury and the location, leads to distinct histological and molecular characteristics. It comes as no surprise that therapeutics to promote improved tendon healing have been met with mixed outcomes. For instance, animal studies have shown that platelet rich plasma improved rotator cuff healing but had a detrimental effect in the FDL. Similarly, augmenting rotator cuff repair with TGF-β3 led to mixed outcomes. Lastly, injections with adipose or mesenchymal stem cells improved some tendon injuries but had a negligible or detrimental effect in others. Data suggests that management for tendinopathy may be most effective by tailoring interventions to the stage of pathology and the associated biological processes. Thus, there is a need for a therapeutic intervention that can promote repair of tendon injuries despite of the wide variability in biological environment associated with different states of injury.

Citation or identification of any document in this application is not an admission that such a document is available as prior art to the present invention.

### SUMMARY

Described in certain example embodiments herein are compositions for treating tendon and/or ligament injury, the composition comprising a decellularized tendon extracellular matrix (ECM) composition comprising one or more bioactive factors having a tendon injury induced bioactive factor profile.

In certain example embodiments, the decellularized tendon ECM composition comprises pulverized decellularized tendon ECM.

In certain example embodiments, the decellularized tendon ECM composition comprises a hydrogel. In certain example embodiments, the hydrogel is PEG-4MAL.

In certain example embodiments, the tendon injury induced bioactive factor profile is a 7-day post injury tendon injury induced bioactive factor profile. In certain example embodiments, the tendon injury induced bioactive factor profile comprises one or more proteins selected from Fibrinogen β chain, fibronectin, Myosin-9, α-Globin 1, ATP synthase subunit β, mitochondrial, Glyceraldehyde-3-phosphate dehydrogenase, Histone H2A type 3, Histone H3.2, Histone H4, Tubulin β-5 chain, Vimentin, periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein S100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof.

In certain example embodiments, one or more of the bioactive factors in the one or more tendon injury induced bioactive factor profile are recombinant proteins. In certain example embodiments, none of the bioactive factors in the one or more tendon injury induced bioactive factor profile are recombinant proteins.

In certain example embodiments, the decellularized tendon composition is prepared from a tendon harvested from a donor about 7 days post tendon injury.

Described in certain example embodiments herein are compositions for treating tendon and/or ligament injury, the composition comprising a protein composition comprising a plurality of bioactive factors, wherein the protein composition comprises a tendon injury induced bioactive factor profile.

In certain example embodiments, the tendon injury induced bioactive factor profile is a 7-day post injury tendon injury induced bioactive factor profile. In certain example embodiments, the tendon injury induced bioactive factor profile comprises one or more proteins selected from Fibrinogen β chain, fibronectin, Myosin-9, α-Globin 1, ATP synthase subunit β, mitochondrial, Glyceraldehyde-3-phosphate dehydrogenase, Histone H2A type 3, Histone H3.2, Histone H4, Tubulin β-5 chain, Vimentin, periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein S100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof.

In certain example embodiments, one or more of the plurality of bioactive factors are isolated from decellularized tendon extracellular matrix (ECM). In certain example embodiments, the decellularized tendon ECM is post-injury decellularized tendon ECM, optionally day 7 post-injury decellularized tendon ECM.

In certain example embodiments, one or more of the plurality of bioactive factors are recombinant proteins. In certain example embodiments, none of the bioactive factors are recombinant proteins.

In certain example embodiments, the composition further comprises a hydrogel. In certain example embodiments, the hydrogel is PEG-4MAL.

Described in certain example embodiments herein are methods of treating a tendon and/or ligament injury, the method comprising a composition to treat tendon injury of any one of the preceding paragraphs and as described elsewhere herein to a subject in need thereof, optionally at the site of a tendon injury.

In certain example embodiments, the composition is effective to increase tendon and/or ligament stiffness, improve tendon matrix alignment, or both.

In certain example embodiments, wherein the tendon injury induced bioactive factor profile is a 7-day post injury tendon injury induced bioactive factor profile, and optionally wherein the a 7-day post injury tendon injury induced bioactive factor profile comprises one or more proteins selected from Fibrinogen β chain, fibronectin, Myosin-9, α-Globin 1, ATP synthase subunit β, mitochondrial, Glyceraldehyde-3-phosphate dehydrogenase, Histone H2A type 3, Histone H3.2, Histone H4, Tubulin β-5 chain, Vimentin, periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein S100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C—Schematic of therapeutic development and treatment. (FIG. 1A) ECM-derived construct development involved patellar tendon isolation, decellularization, and tissue pulverization. Pulverized tissue was then encapsulated within a PEG-4MAL hydrogel and delivered into B6 hosts immediately following a 1-mm midsubstance punch injury. (FIG. 1B) ECM-derived constructs were developed from MRL/MpJ or B6 uninjured ECM, 3-day-provisional ECM, or 7-day-provisional ECM. (FIG. 1C) Injured B6 tendons treated with ECM-derived constructs were submitted to structural, mechanical or histological analysis at 1 or 6 W to assess the efficacy of these constructs at modulating the canonical tendon healing response.

FIG. 2A-2B— Matrix disorganization in B6 injured tendons at (FIG. 1A) 1 week (1 W) and (FIG. 1B) 6 weeks (6 W) following treatment with PEG-4MAL vehicle alone or PEG-4MAL in addition to ECM-derived therapeutic treatment from MRL/MpJ Uninjured ECM (MU), MRL/MpJ 3-day-provisional-ECM (M3), MRL/MpJ 7-day-provisional-ECM (M7), B6 Uninjured ECM (BU), B6 3-day-provisional-ECM (B3), or B6 7-day-provisional-ECM (B7). Tendons treated with M7 showed improvements in matrix alignment compared to injured-untreated controls at 6 W. Dashed line represents mean matrix disorganization for injured-untreated controls. #P≤0.1, *P≤0.05.

FIG. 3A-3C— Mechanical assessment of injured B6 patellar tendons after 6-weeks (6 W) post-treatment with vehicle alone or vehicle with ECM-derived therapeutic constructs. (FIG. 3A) M7-treated tendons showed increased stiffness while treatment with PEG-4MAL alone exhibited decreased stiffness compared to injured-untreated controls. (FIG. 3B) No differences were found in the ultimate load of treated tendons compared to injured-untreated controls. (FIG. 3C) No differences were found in the stress relaxation of treated tendons compared to injured-untreated controls. Dashed line represents mean mechanical data for injured-untreated controls. #P≤0.1,*P≤0.05. B3, B6 3-day-provisional-ECM; B7, B6 7-day-provisional-ECM; BU, B6 in injured ECM; M3, MRL/MpJ 3-day-provisional-ECM; M7, MRL/MpJ 7-day provisional ECM; MU, MRL/MpJ uninjured ECM.

FIG. 4A-4C—Assessment of collagen III content. (FIG. 4A-4B) M7-treated B6 tendons exhibited a decrease in the percent area of the injured region that stained positive for collagen III content compared to injured-untreated controls at both 1-week (1 W) and 6-weeks (6 W) post-treatment. (FIG. 4C) Representative analysis of histological sections. #P≤0.1,*P≤0.05. (B6 tendons treated with MRL/MpJ 7-day-provisional-ECM [M7]). Scale bar=200 μm.

FIG. 5A-5C—Glycosaminoglycan (GAG) content assessment via toluidine blue staining. (FIG. 5A) No differences were found after 1 week (1 W). (FIG. 5B) M7-treated tendons exhibited a decrease in GAG content compared to injured-untreated controls at 6-weeks (6 W) post-treatment. (FIG. 5C) Representative histological sections at 1 W and 6 W. *p≤0.05. (B6 tendons treated with MRL/MpJ 7-day-provisional-ECM [M7]). Scale bar=200 μm.

FIG. 6A-6C—Assessment of cell morphology. (FIG. 6A-6B) M7-treated tendons exhibited a more elongated cell morphology compared to injured-untreated controls at 1-week (1 W) and 6-weeks (6 W) post-treatment. (FIG. 6C) Representative histological sections at 1 W and 6 W. #P≤0.1, *P≤0.05. (B6 tendons treated with MRL/MpJ 7-day-provisional-ECM [M7]). Green arrows point to elongated cells, while red arrows point to more rounded cells. Scale bar=50 μm

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used interchangeably herein, “profile” and “signature” refer to a set of gene or genes, protein or proteins, or epigenetic element(s) whose expression or whose occurrence is associated with a specific cell, tissue or tissue component, or organ type, subtype, state, and/or condition. Such a profile can therefore be used to determine an identity, condition, functionality(ies), and/or characteristic(s), of a cell(s), tissue(s), organ(s), composition, formulation, and/or the like. Profiles can also be used to quantify the number of cells of a particular type or subtype present in in a population of cells. Methods and techniques for determining the presence, amount, and/or change in amounts and/or presence of genes and proteins and expression thereof and epigenetic aspects are generally known in the art and can be used to interrogate a profile of a cell(s), tissue(s), organ(s), or other composition or formulation. A profile can be exclusive to a specific cell, tissue or tissue component, or organ type, subtype, state, and/or condition. A profile can also refer to any set of up- and down-regulated genes and/or gene products (e.g., RNA transcripts and proteins) that are representative of or specific to a cell, tissue or tissue component, or organ type, subtype, state, and/or condition. A profile can be composed of any number of genes, proteins epigenetic elements, and/or combinations thereof. For example, a gene signature may include a list of genes differentially expressed in a distinction of interest. The signature can be composed completely of or contain 1, 2, 3, 4, 5, 6, 7, 8, 9, 10, 11, 12, 13, 14, 15, 16, 17, 18, 19, or 20 or more genes, proteins and/or epigenetic elements. In aspects, the signature can be composed completely of or contain 1-20 or more, 2-20 or more, 3-20 or more, 4-20 or more, 5-20 or more, 6-20 or more, 7-20 or more, 8-20 or more, 9-20 or more, 10-20 or more, 11-20 or more, 12-20 or more, 13-20 or more, 14-20 or more, 15-20 or more, 16-20 or more, 17-20 or more, 18-20 or more, 19-20 or more, or 20 or more genes, proteins and/or epigenetic elements. Exemplary profiles demonstrative of the present disclosure are described in further detail elsewhere herein.

As used herein, “tendon injury induced bioactive factor profile” refers to a profile that is specific to tendons or component thereof (such as the extracellular matrix or a decellularized extracellular matrix produced from the extracellular matrix) that have been injured. Injury can be from any source such as trauma, disease, or degeneration. Likewise, as used herein a “n day post-injury tendon injury induced bioactive profile” refers to a profile from an injured tendon or component thereof that is n days post injury. n can be any number. In some embodiments, n is 1, 2, 3, 4, 5, 6, 7, 8, 9, 10, 11, 12, 13, or 14.

As used herein, “hydrogel” refers to a gelatinous colloid, or aggregate of polymeric molecules in a finely dispersed semi-solid state, where the polymeric molecules are in the external or dispersion phase and water (or an aqueous solution) is forms the internal or dispersed phase. Generally, hydrogels are at least 90% by weight of an aqueous solution, however, it will be appreciated that a hydrogel of the present disclosure may be other percent aqueous solution.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours, days, weeks, months, or years.

As used herein, the term “recombinant” or “engineered” generally refers to a non-naturally occurring nucleic acid, nucleic acid construct, peptide or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic effects. A “least effective amount”, as used herein, refers to the lowest amount needed to achieve one or more of the desired results.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Tendons are comprised of a host population of elongated tenocytes, as well as numerous other cell types such as chondrocyte-like cells, and tendon progenitor cells residing in a highly aligned extracellular matrix (ECM) that is responsible for providing mechanical integrity during loading. Following injuries, mammalian adult tendons heal by scar deposition at the injury site, represented by a disorganized structural matrix, altered tissue composition, loss of mechanical integrity, in-creased cellularity and a shift in cell morphology towards a rounded pathological phenotype. To improve the aberrant healing response following tendinopathies, therapeutics that are based on principles from embryonic healing, mechanical stimulation, specific growth factor delivery and synthetic tissue engineering have been utilized to identify potential targets to encourage tissue regeneration. However, while these treatments have provided valuable insight into the tendon healing response, their interrogation of isolated components necessary for a healthy tendon environment do not adequately emulate the biochemical complexity of the ECM necessary to stimulate improved healing.

Utilizing naturally occurring ECM-derived therapeutics has be-come an attractive approach because of their ability to integrate complex networks of proteins into the injury site, thereby promoting recovery of the structural and compositional properties of native tissues. Accordingly, ECM-derived scaffolds have been used to improve acute injuries in vivo in skeletal muscle and heart. Still, while ECM scaffolds have become a promising tool in regenerative medicine, the use of these constructs to treat tendon injuries has reached limited, if any, clinical relevancy.

With that said, embodiments disclosed herein provide compositions to treat tendon and/or ligament injuries (e.g., lesions) that includes one or more of bioactive factors, wherein the one or more bioactive factors has a tendon injury induced bioactive factor profile, optionally a 7-day post injury tendon injury induced bioactive factor profile. In some embodiments, the composition includes decellularized tendon extracellular matrix, a hydrogel, or both. In some embodiments, the hydrogel is PEG-4MAL. The composition can administered to a subject in need thereof, optionally at the site of a tendon and/or ligament injury. In some embodiments, the composition can improve tendon and/or ligament healing, such as by improving one or more aspects of tendon and/or ligament structure, function, or both. In some embodiments, the composition improves tendon and/or ligament stiffness, matrix alignment, or both. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Compositions for Treating Tendon Injury

Described in several embodiments herein are compositions that can be useful for treating tendon injury. As demonstrated in the working examples herein, bioactive factor containing compositions having a post-injury tendon profile can be effective to improve an attribute of injured tendons. In some embodiments, the bioactive factor profile of the compositions can recapitulate the bioactive factor profile of a decellularized post-injury tendon extracellular matrix (ECM). The compositions can include decellularized tendon extracellular matrix, optionally post-injury decellularized tendon extracellular matrix and/or a hydrogel. Further compositions, embodiments, and features are discussed below and elsewhere herein.

Described in certain example embodiments are compositions for treating tendon and/or ligament injury that include a decellularized tendon extracellular matrix (ECM) composition comprising one or more bioactive factors having a tendon injury induced bioactive factor profile. In some embodiments, the decellularized tendon ECM is pulverized, morselized, pelleted, granulated, or any combination thereof. In certain example embodiments, the decellularized tendon ECM composition comprises pulverized decellularized tendon ECM. In some embodiments, the one or more bioactive factors are bound to or otherwise associated with the decellularized ECM.

In certain example embodiments, the decellularized tendon ECM composition comprises a hydrogel. In some embodiments, the decellularized ECM, the one or more bioactive factors, or both are bound to, imbedded in, or otherwise associated with the hydrogel. The hydrogel can be natural, synthetic, or a combination thereof. In some embodiments, the hydrogel is durable (i.e., non-degradable). In some embodiments, the hydrogel is degradable, optionally biodegradable. In some embodiments, the hydrogel can be responsive to a stimuli, which can be internal to a body of a subject or external to the body of a subject. Exemplary stimuli include, but are not limited to temperature, pH, ionic strength, radiation, metal, electric field, ultrasound, urea, glucose magnetic field, light, etc. In certain embodiments, the hydrogel is a bioactive hydrogel. In some embodiments, the hydrogel is cationic, anionic, neutral, or ampholytic. In some embodiments, the hydrogel is amorphous or semicrystalline. The hydrogels can be prepared by any suitable method or technique. Crosslinking of polymers to form a hydrogel can be accomplished by any suitable method, including, but not limited to, physical crosslinking (e.g., polyelectrolyte complexation, hydrogen bonding, and/or hydrophobic association), chemical cross-linking, free radical polymerization, or irradiation crosslinking. In some embodiments, the hydrogel is a homopolymer hydrogel. In some embodiments, the hydrogel is a copolymer hydrogel. In some embodiments, the hydrogel is a semi-interpenetrating network. In some embodiments, the hydrogel is an injectable hydrogel. See e.g., El-Sherbiny and Yacoub. Glob Cardiol Sci Pract. 2013; 2013(3): 316-342; Lee and Mooney. 2001. Chem Rev. 101(7):1869-1880; Manta et al., Materials (Basel). 2019 October; 12(20): 3323; Li et al., Front. Chem., 22 Oct. 2018|https://doi.org/10.3389/fchem.2018.00499; Liu et al., Front. Chem., 6 Mar. 2020|https://doi.org/10.3389/fchem.2020.00124; Liu et al., J Tissue Eng Regen Med. 2020 September; 14(9):1333-1348. doi: 10.1002/term.3078; Farnebo et al., Tissue Eng Part A. 2014 May; 20(9-10):1550-61. doi: 10.1089/ten.TEA.2013.0207; Stoppato et al., Gels Handbook, pp. 271-293 (2016); Silva et al., Journal of Nanobiotechnology volume 18, Article number: 23 (2020); Gomez-Florit et al., Molecules 2020, 25(24), 5858; Yang et al., Sci. Adv. 2021. 7(26) DOI: 10.1126/sciadv.abg3816; Xu et al., Biomater. Sci., 2021, 9, 1237-1245; which are incorporated by reference as if expressed in their entireties herein and can be adapted for use with the present description herein.

Exemplary hydrogels include, but are not limited to those, formed from hyaluronic acid and derivatives thereof, peptide amphiphile-Ti composite, chondroitin sulfate, gelatin, fibrin, alginate, collogen, carboxymethylcellulose, dextran, chitin, hydroxyapatite, agarose carbomer, poly(ethylene glycol) (PEG), poly(lactic acid) (PLA), PEG-PLA, poly(ethylene glycol) diacrylate (PEGDA), poly(vinyl alcohol) (PVA), poly(ethylene oxide) (PEO), Semi-interpenetrating polymeric network, MA, gel-MA, poly(hydroxyl-ethyl methacrylate) (PHEMA), methacrylated dextran-graft-lysine (Dex-MA-LA), maleimide, PEG-maleimide, PEG-4MA, methacrylamide-modified gelatin (Gel-MA), self-assembling peptides (SAPs) and any combination thereof. See e.g., El-Sherbiny and Yacoub. Glob Cardiol Sci Pract. 2013; 2013(3): 316-342; Lee and Mooney. 2001. Chem Rev. 101(7):1869-1880; Manta et al., Materials (Basel). 2019 October; 12(20): 3323; Li et al., Front. Chem., 22 Oct. 2018|https://doi.org/10.3389/fchem.2018.00499; Liu et al., Front. Chem., 6 Mar. 2020|https://doi.org/10.3389/fchem.2020.00124; Liu et al., J Tissue Eng Regen Med. 2020 September; 14(9):1333-1348. doi: 10.1002/term.3078; Farnebo et al., Tissue Eng Part A. 2014 May; 20(9-10):1550-61. doi: 10.1089/ten.TEA.2013.0207; Stoppato et al., Gels Handbook, pp. 271-293 (2016); Silva et al., Journal of Nanobiotechnology volume 18, Article number: 23 (2020); Gomez-Florit et al., Molecules 2020, 25(24), 5858; Yang et al., Sci. Adv. 2021. 7(26) DOI: 10.1126/sciadv.abg3816; Xu et al., Biomater. Sci., 2021, 9, 1237-1245; which are incorporated by reference as if expressed in their entireties herein and can be adapted for use with the present description herein.

In some embodiments, the hydrogel is a PEG-maleimide hydrogel. See e.g., Garcia, A. J. Ann Biomed Eng. 2014 February; 42(2): 312-322. In certain example embodiments, the hydrogel is a PEG-4MAL hydrogel (see e.g., Cruz-Acuña, et a. Nature Protocols. 13:2102-2119 (2018)).

In certain example embodiments, the tendon injury induced bioactive factor profile is a 7-day post injury tendon injury induced bioactive factor profile. In some embodiments, the tendon injury induced bioactive profile includes 1-100 or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to/or 100 or more proteins.

In certain example embodiments, the tendon injury induced bioactive factor profile includes or is composed only of one or more proteins selected from Fibrinogen β chain, fibronectin, Myosin-9, α-Globin 1, mitochondrial ATP synthase subunit β, Glyceraldehyde-3-phosphate dehydrogenase, Histone H2A type 3, Histone H3.2, Histone H4, Tubulin β-5 chain, Vimentin, periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein S100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof.

In certain example embodiments, the tendon injury induced bioactive factor profile includes or is composed only of one or more proteins selected from Fibrinogen β chain, fibronectin, Myosin-9, α-Globin 1, mitochondrial ATP synthase subunit β, Glyceraldehyde-3-phosphate dehydrogenase, Histone H2A type 3, Histone H3.2, Histone H4, Tubulin β-5 chain, Vimentin, and any combination thereof.

In certain example embodiments, the tendon injury induced bioactive factor profile includes or is composed only of one or more proteins selected from periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein S100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof. In certain example embodiments, one or more of the bioactive factors in the composition are recombinant (or engineered) proteins. In some embodiments, all of the bioactive factors are recombinant proteins. In certain example embodiments, none of the bioactive factors in the composition are recombinant proteins.

The composition can be prepared by harvesting tendon from a donor and preparing decellularized tendon ECM from the harvested donor. In some embodiments, the tendon is harvested from a donor at n day(s) post-tendon injury, where n is about 1, 2, 3, 4, 5, 6, 7, 8, 9, 10, 11, 12, 13, 14, or more days. In certain example embodiments, the decellularized tendon composition is prepared from a tendon harvested from a donor about 7 days post tendon injury.

Described in certain example embodiments herein are compositions for treating tendon injury, the composition comprising a protein composition comprising a plurality of bioactive factors, wherein the protein composition comprises a tendon injury induced bioactive factor profile. Tendon injury bioactive factor profiles are as previously described. In some embodiments, the tendon injury induced bioactive factor profile is 7-day post injury tendon injury induced bioactive factor profile. 7-day post injury tendon injury induced bioactive factor profiles are as previously described.

In certain example embodiments, one or more of the plurality of bioactive factors are isolated from decellularized tendon extracellular matrix (ECM). Decellularized tendon ECM and methods of decellularization are as described elsewhere herein. In certain example embodiments, the decellularized tendon ECM is post-injury decellularized tendon ECM, optionally day 7 post-injury decellularized tendon ECM. Post-injury decellularized tendon ECM is as described elsewhere herein. In some embodiments, the composition does not contain ECM or decellularized tendon ECM. Any suitable method of tendon ECM decellularization can be used. In some embodiments, a detergent based ECM decellularization method is used. See e.g., Paredes et al., FASEB. 2020; 1-16 and Working examples herein.

In certain example embodiments, one or more of the plurality of bioactive factors are recombinant proteins. In certain example embodiments, all of the plurality of bioactive factors are recombinant proteins. In certain example embodiments, none of the bioactive factors are recombinant proteins. In certain example embodiments, one or more of the plurality of bioactive factors are natural proteins. In certain example embodiments, none of the plurality of bioactive factors are natural proteins. In certain example embodiments, all of the plurality of bioactive factors are natural proteins.

In certain example embodiments, the composition further includes a hydrogel. Exemplary hydrogels are as described elsewhere herein. In some embodiments, the hydrogel is a PEG-maleimide hydrogel. See e.g., Garcia, A. J. Ann Biomed Eng. 2014 February; 42(2): 312-322. In certain example embodiments, the hydrogel is a PEG-4MAL hydrogel (see e.g., Cruz-Acuña, et a. Nature Protocols. 13:2102-2119 (2018)). In some embodiments, the composition does not contain a hydrogel.

Where a hydrogel and/or decellularized ECM is present in the composition, the bioactive factors can be bound with or otherwise associated or trapped within the decellularized ECM and/or hydrogel.

In certain embodiments, the composition is effective to treat a tendon injury (e.g., a tendon lesion). In some embodiments the composition is effective to promote, stimulate and/or otherwise improve healing of a tendon post-injury. In some embodiments, the composition is effective to improve a structure, a function, or other characteristic of a tendon that is otherwise damaged, reduced, or lost due to injury. In some embodiments, the composition is effective to increase stiffness of a tendon, improve matrix alignment, or both. In some embodiments, stiffness is increased by 0.01 to 1,000 percent or more, such as 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 100%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, 500%, 505%, 510%, 515%, 520%, 525%, 530%, 535%, 540%, 545%, 550%, 555%, 560%, 565%, 570%, 575%, 580%, 585%, 590%, 595%, 600%, 605%, 610%, 615%, 620%, 625%, 630%, 635%, 640%, 645%, 650%, 655%, 660%, 665%, 670%, 675%, 680%, 685%, 690%, 695%, 700%, 705%, 710%, 715%, 720%, 725%, 730%, 735%, 740%, 745%, 750%, 755%, 760%, 765%, 770%, 775%, 780%, 785%, 790%, 795%, 800%, 805%, 810%, 815%, 820%, 825%, 830%, 835%, 840%, 845%, 850%, 855%, 860%, 865%, 870%, 875%, 880%, 885%, 890%, 895%, 900%, 905%, 910%, 915%, 920%, 925%, 930%, 935%, 940%, 945%, 950%, 955%, 960%, 965%, 970%, 975%, 980%, 985%, 990%, 995%, to/or 1000% or more. Exemplary methods of measuring an effective, such as those described herein, of the composition are described in greater detail elsewhere herein and are generally known in the art.

### Pharmaceutical Formulations

In some embodiments, an amount composition for tendon and/or ligament injury of the present description herein is included in a pharmaceutical formulation, such as a primary active agent of the pharmaceutical formulation. In some embodiments, the composition is provided in the formulation as an effective amount (such as a therapeutically effective amount) or least effective amount. The pharmaceutical formulation can contain one or more secondary active agents and/or inert ingredients, such as carriers and excipients. Where appropriate a compound present in the pharmaceutical formulation can be provided as a pharmaceutically acceptable salt.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

### Pharmaceutically Acceptable Carriers and Secondary Ingredients and Agents

The pharmaceutical formulation can include, where appropriate, a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combination thereof.

In some embodiments, the pharmaceutical formulation further comprises cells, such as stem cells. Exemplary stem cells include, but are not limited to, mesenchymal stem cells, embryonic stem cells, adipose stem cells, tendon stem cells, or any combination thereof.

In some embodiments, the pharmaceutical formulation comprises exosomes, optionally tendon derived exosomes. See e.g., Zhang et al., Stem Cell Research & Therapy volume 11, Article number: 402 (2020).

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. In some embodiments, the one or more effects of e.g., the primary active agent in the formation is healing a tendon injury are (e.g., a tendon lesion); promoting, stimulating and/or otherwise improve healing of a tendon post-injury; improving a structure, a function, or other characteristic of a tendon that is otherwise damaged, reduced, or lost due to injury. In some embodiments, the therapeutic effect is to increase stiffness of a tendon, improve matrix alignment, or both. In some embodiments, the therapeutic effect is to increase stiffness by 0.01 to 1,000 percent or more or a value or range thereof as discussed above in relation to the compositions. Exemplary methods of measuring an effective, such as those described herein, of the composition are described in greater detail elsewhere herein and are generally known in the art.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the percent of primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, intratendinous, parenteral, subcutaneous, intramuscular, intravenous, internasal, and intradermal. Other appropriate routes are described elsewhere herein. Such formulations can be prepared by any method known in the art.

In some embodiments, the dosage form is adapted for delivery to a tendon (or intratendinous delivery). In some embodiments, the dosage form is adapted for delivery to a ligament. In some embodiments, such a dosage form is adapted for injection, such as to a joint, tendon, or ligament. Dosage forms for injection are described in greater detail elsewhere herein.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof. Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combination thereof.

In some embodiments, the co-therapy further comprises providing cells, such as stem cells to the subject, optionally at the site of tendon injury. Exemplary stem cells include, but are not limited to, mesenchymal stem cells, embryonic stem cells, adipose stem cells, tendon stem cells, or any combination thereof.

In some embodiments, the co-therapy includes exosomes, optionally tendon derived exosomes, provided to the subject. See e.g., Zhang et al., Stem Cell Research & Therapy volume 11, Article number: 402 (2020).

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, cells, and/or the like described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet, gel, liquid, powder, and/or the like) or in separate formulations. When the compounds, compositions, formulations, and cells and/or the like described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof can be in need of a treatment of a tendon injury or lesion and/or ligament injury.

### Methods of Treating Tendon Injury

The compositions and formulations described herein can be used to treat a tendon injury in a subject in need thereof. Generally, such methods include administering, to the subject in need thereof, a composition of the present disclosure described elsewhere herein. Administration can be intratendinous, optionally at the site of tendon injury or lesion. Administration can be to a ligament, optionally at the site of a ligament injury or lesion. Other suitable route are described in greater detail elsewhere herein, and include without limitation, intravenous, intrasynovial, intrarticular, intramuscular, and subcutaneous.

The compositions and formulations can be administered one or more (e.g., 1, 2, 3, 4, 5, 6, 7, 8, 9, 10 or more) times per day, week, month, or year. In some embodiments, a loading amount may be delivered one or more times over a period of time ranging from 1-365 days or more followed by one or more maintenance doses over a period of 1, 2, 3, 4, 5, 6, 7, 8, 9, 10 or more days, weeks, months or years. A loading dose is typically, but not always, higher in amount of the active ingredient(s) than the maintenance dose. In some embodiments, the “loading dose” is the same number of the active agent(s) as maintenance doses but the loading doses are given more frequently during a “loading period of time” than during maintenance. Without being bound by theory, it is believed that dose loading increases the amount of the active agents to at or above a threshold amount in the subject or a location within a subject so as to obtain a therapeutic effect, which then can be maintained by subsequent maintenance doses.

In some embodiments, the method includes delivering to the subject in need thereof a co-therapy. In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combination thereof.

In some embodiments, the co-therapy further comprises providing cells, such as stem cells to the subject, optionally at the site of tendon injury. Exemplary stem cells include, but are not limited to, mesenchymal stem cells, embryonic stem cells, adipose stem cells, tendon stem cells, or any combination thereof.

In some embodiments, the co-therapy includes exosomes, optionally tendon derived exosomes, provided to the subject. See e.g., Zhang et al., Stem Cell Research & Therapy volume 11, Article number: 402 (2020).

Co-therapies can be the different therapies or formulations and can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

In certain embodiments, the composition is effective to treat a tendon injury (e.g., a tendon lesion). In some embodiments the composition is effective to promote, stimulate and/or otherwise improve healing of a tendon post-injury. In some embodiments, the composition is effective to improve a structure, a function, or other characteristic of a tendon that is otherwise damaged, reduced, or lost due to injury. In some embodiments, the composition is effective to increase stiffness of a tendon, improve matrix alignment, or both. In some embodiments, the composition is effective to increase stiffness by 0.01 percent to 1000 percent or more or by any amount previously described with respect to the compositions.

In some embodiments, the compositions and/or formulations thereof described herein are delivered to a tendon or a ligament during a surgical procedure and/or repair. In some embodiments, the compositions and/or formulations are delivered to a tendon or a ligament non-surgically via injection to the tendon and/or ligament.

Exemplary methods of measuring an effective, such as those described herein, of the composition are described in greater detail elsewhere herein and are generally known in the art.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Introduction

Tendons are comprised of a host population of elongated tenocytes, as well as numerous other cell types such as chondrocyte-like cells, and tendon progenitor cells residing in a highly aligned extracellular matrix (ECM) that is responsible for providing mechanical integrity during loading.¹ Following injuries, mammalian adult tendons heal by scar deposition at the injury site, represented by a disorganized structural matrix, altered tissue composition, loss of mechanical integrity, in-creased cellularity and a shift in cell morphology towards a rounded pathological phenotype.²⁻⁴ To improve the aberrant healing response following tendinopathies, therapeutics that are based on principles from embryonic healing, mechanical stimulation, specific growth factor delivery and synthetic tissue engineering have been utilized to identify potential targets to encourage tissue regeneration.⁵⁻⁹ However, while these treatments have provided valuable insight into the tendon healing response, their interrogation of isolated components necessary for a healthy tendon environment do not adequately emulate the biochemical complexity of the ECM necessary to stimulate improved healing.

Utilizing naturally occurring ECM-derived therapeutics has be-come an attractive approach because of their ability to integrate complex networks of proteins into the injury site, thereby promoting recovery of the structural and compositional properties of native tissues.¹⁰ Accordingly, ECM-derived scaffolds have been used to improve acute injuries in vivo in skeletal muscle and heart.^{11,12} Still, while ECM scaffolds have become a promising tool in regenerative medicine, the use of these constructs to treat tendon injuries has been limited, and therefore the optimal composition to develop ECM derived therapeutics remains to be elucidated.

Interestingly, the Murphy Roths Large (MRL/MpJ) mouse has recently been identified as a model of adult mammalian scarless tendon healing.¹³⁻¹⁵ More specifically, MRL/MpJ tendon healing is characterized by an early deviation in ECM composition post-injury followed by improved mechanical, structural and cellular behavior in vivo, compared to canonically healing C57Bl/6 (B6). Identification of the improved healing capacity of MRL/MpJ tendons provides a model wherein the synergistic behavior of multiple biochemical factors that lead to re-generation could be interrogated. In this manner, Applicant has previously utilized organ culture to determine that the innate tendon properties of the MRL/MpJ act as a driver of its improved healing response even when isolated from the systemic environment.¹⁶ Additionally, Applicant has identified that the compositional properties of the ECM obtained from

MRL/MpJ tendons after 7-days post-injury (M7), harness the necessary biochemical cues to improve the proliferative and morphological behavior of canonically healing cells in vitro.¹⁶ However, while these findings highlight the potential role of the innate MRL/MpJ ECM in modulating cell behavior of a non-regenerative model following tendinopathies; the ability of these constructs to stimulate improved tendon healing in vivo remains unknown.

Moreover, while Applicant has identified the ability of M7 constructs to modulate the behavior of scar-mediated healing B6 cells in vitro, the ECM deposited during the beginning of the proliferative stage of healing, after 3-days post-injury, contains a bioactive cocktail of growth factors, cytokines, and glycosaminoglycans (GAGs) that are transiently remodeled throughout the healing response, towards the deposition of larger structural proteins seen after 7 days.^{17,18} Thus, to assess the in vivo therapeutic potential of these two distinct ECM environments, Applicant has developed MRL/MpJ and B6 decellularized ECM-constructs from matrix deposited after 3 and 7-days post-injury. Applicant hypothesized that the unique composition of MRL/MpJ ECM-derived therapeutics harnesses the biological cues to drive an improved structural and functional healing response in B6 mice by 6-weeks post-injury. Consequently, the objectives of this study are to (1) identify an ECM-derived therapeutic platform that leads to the best improvement in the structural, compositional, cellular and mechanical function of scar mediated healing and (2) identify the compositional profile responsible for providing the biochemical cues that harness the ability to improve tendon healing in vivo.

Applicant assessed the long-term structural and mechanical integrity of B6 tendons treated with the MRL/MpJ and B6 derived ECM constructs following a 1-mm acute midsubstance punch injury. Applicant utilized tendon matrix alignment and mechanical properties to identify the most effective ECM construct and subsequently isolated this treatment group for further analysis of its therapeutic effect on healing tendon composition of collagen III and GAGs, which are significantly increased during the scar mediated canonical healing response.18,19 Moreover, cell elongation, which is characteristic of healthy tendon cells, was also assessed and compared to injured-untreated tendons. Lastly, to elucidate the unique compositional cocktail responsible for the improved tendon healing response following treatment, the proteomic profile was obtained for the most and least effective therapeutic constructs. Comparison of the protein composition of the most effective ECM with the one found to be least effective will further inform a compositional template for future development of tissue engineering therapeutics.

### Methods

### Patellar Tendon Punch Injury and Sample Dissection

Under IACUC approval, 16-week-old mice (n=227 Jackson Laboratories, ME) (n=57 bred in-house with sire and dame from Jackson Laboratories, ME) were placed under isoflurane, (2% by volume, 0.3 L/min). Briefly, a skin incision exposed the left patellar tendon of MRL/MpJ (n=52) and B6 (n=232) mice. A medial and lateral incision on the tendon edges was introduced utilizing an 11-blade scalpel to allow for a polyurethane coated stainless-steel backing to be positioned below the patellar tendon. Following, a 1-mm biopsy punch defect was created in the tendon midsubstance.¹³ The backing was removed and skin sutured utilizing 6-0 Prolene sutures. Analgesic was administered, (Buprenorphine, 0.2 mg/kg) and mice resumed cage activity (12-hour light cycle, unlimited access to food/water). Mice were kept under standard housing conditions and sacrificed at 3-days, 1-week, or 6-weeks post-injury via CO2.13

### Development of Therapeutics and PEG-4MAL Hydrogel Delivery System

MRL/MpJ and B6 mice were sacrificed after 3 (n=24,24) or 7 days (n=24,24) post-injury. The midsubstance region from their left injured and right uninjured patellar tendons was collected. Following dissection, tissue was frozen in liquid nitrogen and stored at −80° C. All ECM samples were decellularized utilizing an adapted previously described protocol.16,20 Briefly, samples were submerged in a 50 nM Latrunculin B (BioVision, Milpitas, Calif.) bath for 2 hours, then submitted to a progression of 0.6 M KCl followed by 1.0 M KI solutions for 2 hours each. 30-minute deionized (DI) H₂O washes occurred between all KCl and KI baths. A 12-hour wash was then performed in DIH2O, after-which samples repeated the KCl, DIH2O, and KI progression. A final 48-hour PBS+1 kU/mL Pierce Universal Nuclease (Thermo Fisher Scientific, Waltham, Mass.) wash was performed. Samples were then lyophilized for 48 hours. All incubations were done under agitation (450 rpm) and all solutions contained 1× Halt™ protease inhibitor. Lyophilized tendons were pulverized utilizing a beadruptor device using 2.8-mm in diameter ceramic beads for 4 cycles at 15 seconds/cycle. Following, 1.4-mm in diameter ceramic beads were utilized for 4 additional cycles at 15 seconds/cycle before being placed in −80° C.

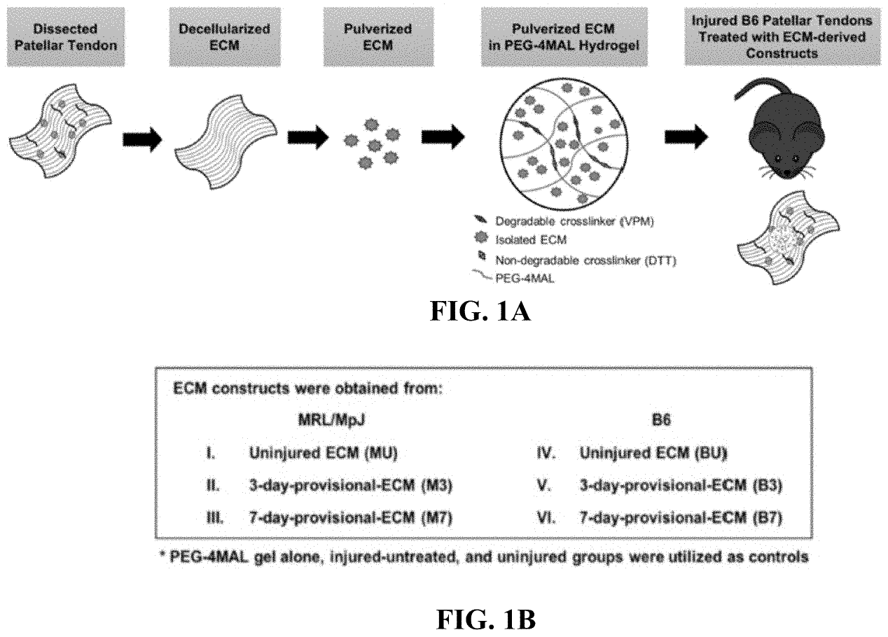

Based on previous studies, 20% (w/v) PEG-4MAL (Laysan Bio Inc, Arab, AL) hydrogels were formed.²¹⁻²³ Briefly, PEG-4MAL was crosslinked with non-degradable dithiothreitol crosslinker and MMP-degradable GCRDVPMSMRGGDRCG peptides (AAPPTec, LLC, Louisville, Ky.) (50% DTT, 50% VPM crosslinker ratio). 200±13 μg of pulverized, B6-3-day-provisional-ECM (B3), B6-7-day-provisional-ECM (B7), MRL/MpJ-3-day-provisional-ECM (M3) or MRL/MpJ-7-day-provisional-ECM (M7) was suspended in the cross-linker solution and combined with the PEG-4MAL to create 2-μ1 hydrogel-therapeutic complexes. To assess whether innate differences in therapeutic potential between naïve MRL/MpJ and B6 ECM ex-tended to the in vivo healing outcome, uninjured MRL/MpJ (MU) and B6 (BU) ECMs were also included in this study and prepared as described above. Hydrogel-therapeutic complexes were inserted into B6 left patellar tendons through the previously described lateral incision introduced to create the 1 mm punch defect. A subset of B6 tendons were left untreated post-injury to comprise the injured-untreated control group, and another was treated with PEG-4MAL delivery system alone to comprise the vehicle-only group. Right limbs were utilized as uninjured controls. Tendons healed for 1 (n=56) or 6 weeks (n=124), before sacrifice, at which point tissues were dissected, flash frozen in liquid nitrogen, and placed at −80° C. until the time of respective analysis. Finally, following sacrifice, mice treated with the ECM-derived constructs were randomly assigned towards structural or mechanical assessment (FIG. 1A-1C).

### Structural Assessment

As previously described, B6 tendons (n=7-8/group) were fixed utilizing a zinc-buffered formalin solution before embedding in paraffin wax and sectioning (10 μm/section).¹⁶ To analyze the auto-fluorescent structural properties of the tendon matrix, unstained sections were imaged via fluorescent microscopy, at ×4 magnification.¹⁶ A custom MATLAB script was used to identify matrix disorganization at the tendon midsubstance for uninjured, treated, and injured-untreated samples.^{13,16}

### Mechanical Testing

Treated B6 tendons, injured-untreated controls, and uninjured controls (n=8/group) were clamped and submerged in a 1×PBS bath at room temperature. Tendons were subjected to an initial load of 0.15 N (Bose ElectroForce 3200 with 10 lb load cell, Bose Corporation, Eden Prairie, Minn.). Preconditioning was then performed (15 cycles at 1% strain, 1 Hz). A 5% strain was applied and held for 300 seconds to assess stress relaxation. Tendons then recovered for 300 seconds under no load. Preconditioning was repeated prior to applying a strain to failure at a rate 0.1% strain/second to assess ultimate load and stiffness.16 Mechanical testing data was collected every 0.05 seconds.

### Selection of the Best and Worst Performing ECM-Derived Therapeutic Constructs

Late stage structural and mechanical assessment at 6-weeks post-injury, were utilized to select the most and least effective ECM-derived therapeutics. The treatment group resulting in the highest improvement compared to injured-untreated controls was deemed the most effective. Due to the high number of groups that did not result in enhanced matrix alignment, stiffness or ultimate load, the group with the lowest overall improvement, as determined by the sample means, was deemed the least effective therapeutic.

Samples treated with the most effective therapeutic were further analyzed to assess deviations in tendon composition and cell behavior compared to injured-untreated controls. Additionally, to identify the compositional profile of the ECM-derived construct that led to improved healing and compare it to the one that had the most detrimental outcomes, the best and worst performing constructs were submitted to proteomic analysis.

### Immunohistochemistry and Histology

Paraffin sections (10 μm/section) (n=7-8/group) were stained with collagen III antibody according to manufacturer's protocol. Briefly, samples were de-paraffinized, rehydrated, placed in Pro-K solution for 3 minutes and blocked (Dako North America, Inc, Carpinteria, Calif.) for 30 minutes at 37° C. Samples were incubated with a collagen III antibody for 1-hour at 37° C. (#AB1832; Chemicon, Temecula, Calif.; 1:500 dilution). Vector-Anti Rabbit secondary (Vector Laboratories, Burlingame, Calif.) was applied for 30 minutes. Diaminobenzidine (DAB) (Sigma-Aldrich, St. Louis, Mo.) was added to visualize the stain. Samples were submerged in Toluidine Blue for 3 minutes as a counter stain. DIH2O washes were performed between all steps.

Collagen III imaging was performed at ×4 magnification via brightfield microscopy. As previously described, RGB images were im-ported into ImageJ and decomposed into Red, Green and Blue chan-nels.24 The Blue channel, representing the IHC signal, was isolated for further analysis. Images were pooled into a stack and a pixel intensity histogram of the merged signal was obtained to establish a threshold for the detection of “on/stained” pixels. The peak of the histogram signal was identified and utilized as this threshold. Values above the threshold intensity were labeled as “on/stained” to reduce the effect of background and shared high intensity pixels from analysis. The mid-substance region of the tendon was isolated and percent stain per area (“on”/total pixels) was measured by a blinded user.

For GAG analysis, additional sections (n=7-8/group) were stained with Toluidine Blue (Electron Microscopy Sciences, Hatfield, Pa.) for 20 minutes. Imaging protocol was performed as described above. Mean intensity measurements of this colorimetric stain were taken to obtain semiquantitative analysis of GAG content. Toluidine stained sections were then imaged at ×40 magnification.13 A square grid was superimposed onto each image utilizing ImageJ, and a random number generator was utilized to select five regions within the grid for cell analysis. Nuclear aspect ratio (major/minor axis) was measured by a blinded user (n=6-8/group).

### Proteomic Analysis of M7 and BU Composition

ECM-derived constructs were homogenized as described (n=4/group). A solution of 400 μL PBS pH 7.4, 6 M Urea, 2 M Thiourea, 10 mM DTT was added for initial digestion. Samples were vortexed (1200 rpm, at room temperature) for 2.5 hours, centrifuged (12 000 g at room temperature) for 5 minutes and supernatant was removed. The insoluble pellet was additionally digested with 400 μL PBS pH 7.4, 6 M Guanidine-Hydrochloride (Gdn-HCL), and 10 mM DTT for 1-hour. Centrifugation and supernatant collection were performed as described. All fractions were concentrated to a volume of 90-100 μL. Sample concentration was determined via Bradford assay, and further quantified by running a precast NOVEX 10% Bis/Tris mini-gel (Invitrogen, Carlsbad, Calif.). Based on the gel quantitation, 4 μg protein for the Urea fractions and 0.8 μg protein for the Gdn-HCL fractions were used for in-solution digestion. Sample volumes were adjusted with 10 mM DTT reducing agent to a final volume of 16 ul for Urea Fractions and 65 ul for Gdn-HCL fractions. The digested fractions for each sample were reconstituted in 20-μL of 0.5% FA for nano-LC-ESI-MS/MS analysis (Orbitrap Fusion™ Tribrid™, Thermo Fisher Scientific, San Jose, Calif.). Proteome Discoverer 2.3 (Thermo Fisher Scientific, Bremen, Germany) was utilized to perform database search using Sequest HT searching engine against MusMusculus Uniprot 2016 database. Data from Urea and Gdn-HCL digestions were pooled for analysis of each individual sample. Samples with at least two unique peptides and ≥2-fold change between groups were included for statistical analysis.25-27 Data obtained from the Proteome Dis-coverer 2.3 Software was used to identify protein classes.

### Statistical Analysis

Data is shown as mean±standard deviation. To assess the effectiveness of ECM-derived constructs at improving the canonical ten-don healing response, a One-way analysis of variance (ANOVA) with Dunnett's multiple comparisons test was utilized to assess structural and mechanical properties of treated groups compared to injured-untreated controls. Additionally, to assess the effectiveness of the ECM-derived constructs at restoring the structure and mechanics of treated tendons to pre-injury levels, groups were compared to un-injured controls using a One-way ANOVA with Dunnett's multiple comparisons test. For histological and cell behavior assessment, in-dividual unpaired student t test were performed between the best performing therapeutic and injured-untreated controls. For proteomic analysis comparing the concentrations of individual proteins between the best and worst performing construct, a non-parametric Kruskal Wallace ANOVA was performed on proteins that were ex-pressed in at least three samples per group. The false discovery rate was controlled post-hoc via a false discovery rate (FDR) corrected method of Benjamini and Hochberg (*q<0.05, #q<0.1). All statistical analysis was performed on raw data prior to normalization.

### Results

Surgery was tolerated and all animals survived until the designated time-point.

### Structural and Functional Assessment of Healing Tendons Following Treatment

Analysis of tendon structure showed that by 6-weeks post-injury matrix

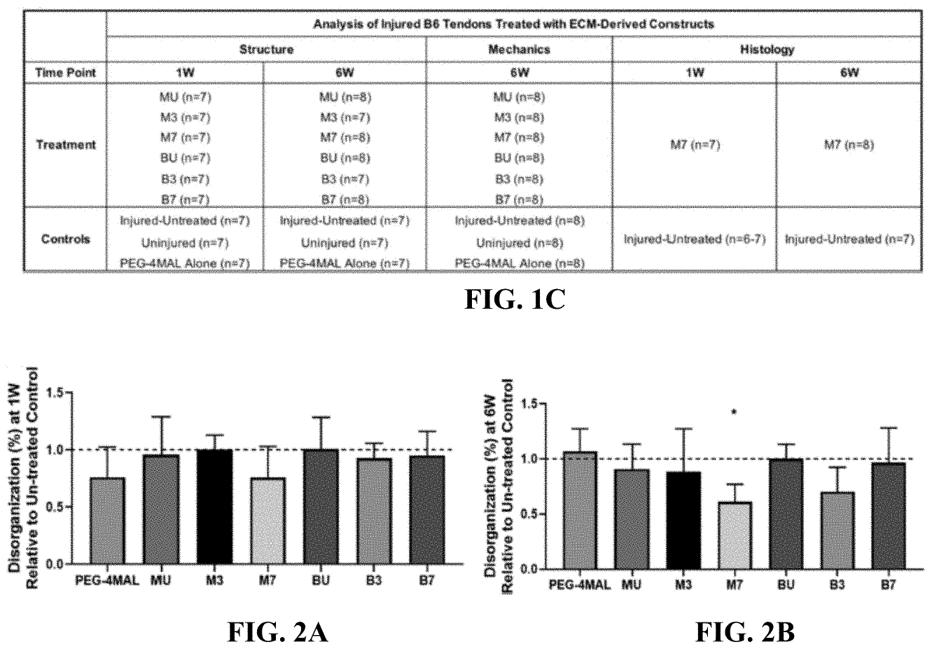

alignment was improved for the M7 (P=0.0205) treated groups compared to injured-untreated controls. No differences were found between treated groups and injured-untreated controls at 1-week post-injury (FIG. 2A-2B).

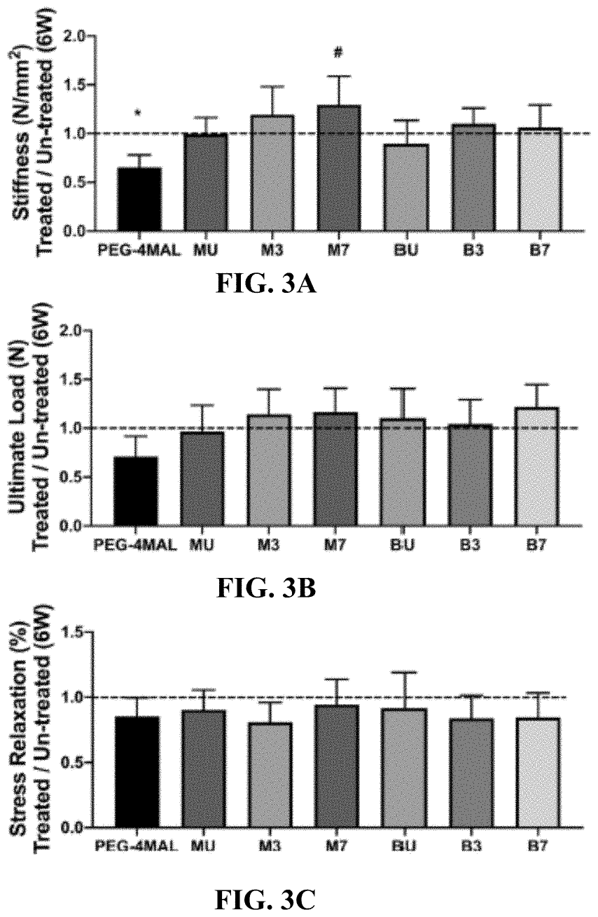

Mechanical assessment showed that only the M7-treated group displayed higher stiffness compared to injured-untreated controls (P=0.0779) by 6 weeks (FIG. 3A). Surprisingly, PEG-4MAL delivery system treatment alone showed significantly lower stiffness compared to injured-untreated controls (P=0.0259). No differences in ultimate load were found between treated groups and injured-untreated controls (FIG. 3B). No differences were found in the stress relaxation of B6 treated tendons compared to injured-untreated controls (FIG. 3C). Lastly, all treated groups exhibited significantly higher levels of matrix disorganization, and significantly lower stiffness and ultimate load than uninjured controls at 6 W (Table 1)(P<0.05 for all groups compared to uninjured).

TABLE 1
Assessment of B6 tendons treated with ECM-derived constructs compared to uninjured controls.

| | Uninjured | PEG-4MAL | MU | M3 | M7 | BU | B3 | B7 |
|---|---|---|---|---|---|---|---|---|
| Matrix | 5.94 ± 5.03 | 78.76 ± 14.9* | 66.8 ± 16.68* | 64.95 ± 28.72* | 44.95 ± 11.7* | 73.75 ± 9.62* | 51.74 ± 16.34* | 71.18 ± 23.08* |
| disorganization | | | | | | | | |
| (%) | | | | | | | | |
| Ultimate load | 7.93 ± 1.68 | 3.13 ± 0.92* | 4.25 ± 1.19* | 5.03 ± 1.13* | 5.13 ± 1.08* | 4.86 ± 1.33* | 4.60 ± 1.10* | 5.36 ± 1.02* |
| (N) | | | | | | | | |
| Stiffness | 43.98 ± 12.67 | 15.36 ± 3.04* | 23.37 ± 4* | 28.13 ± 6.73* | 30.51 ± 6.88* | 21.06 ± 5.67* | 25.88 ± 3.82* | 25.01 ± 5.46* |
| (N/mm²) | | | | | | | | |
| Stress | 24.7 ± 4.80 | 25.41 ± 4.23 | 26.9 ± 4.56 | 24.05 ± 4.51 | 28.11 ± 5.82 | 27.25 ± 8.21 | 24.98 ± 5.31 | 25.13 ± 5.67 |
| relaxation (%) | | | | | | | | |

Note:
All groups exhibited significantly higher matrix disorganization, and lower stiffness and ultimate load than uninjured controls at 6 W regardless of treatment.
Abbreviations: B3, B6 3-day-provisional-ECM; B7, B6 7-day-provisional-ECM; BU, B6 Uninjured ECM; M3, MRL/MpJ 3-day-provisional-ECM; M7, MRL/MpJ 7-day-provisional-ECM; MU, MRL/MpJ uninjured ECM.
*Significant difference from uninjured control.

### Selection of the Best and Worst Performing ECM-Derived Therapeutic Constructs

M7 was identified as the most effective therapeutic since tendons treated with these constructs were the only group that exhibited both improved stiffness (P=0.0779) and matrix alignment (P=0.0205) compared to injured-untreated controls. Furthermore, BU was identified as the least effective therapeutic, since treatment with this construct resulted in the highest degree of matrix disorganization and lowest stiffness based on group means (Table 2).

TABLE 2
Selection of the least effective therapeutic based on
group means of treatment groups (MU, M3, BU, B3, and
B7) that did not show statistical improvements compared
to injured-untreated controls in matrix alignment, stiffness
and ultimate load after 6-weeks post-treatment

| | Matrix | | Ultimate |
|---|---|---|---|
| ECM-derived | disorganization | Stiffness | load |
| Constructs | (%)* | (N/mm²)* | (N)* |
| B3 | 51.74 | 25.88 | 4.60 |
| M3 | 64.95 | 28.13 | 5.03 |
| MU | 66.9 | 23.37 | 4.25 |
| B7 | 71.18 | 25.01 | 5.36 |
| BU | 73.75 | 21.06 | 4.86 |

Note:
BU was found as the least effective treatment.
Abbreviations: B3, B6 3-day-provisional-ECM; B7, B6 7-day-provisional-ECM; BU, B6 uninjured ECM; M3, MRL/MpJ 3-day-provisional-ECM; MU, MRL/MpJ uninjured ECM.
*Mean values per group.

Compositional and Cell Behavior Assessment of Healing Tendons Following Treatment with M7 Construct

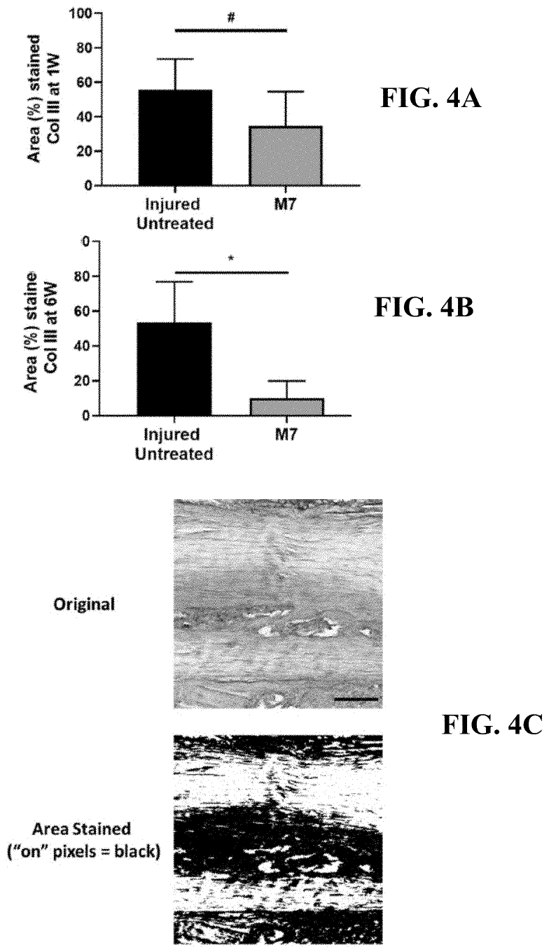

There was a decrease in the percent area of the injured region that stained positive for collagen III content in the healing M7-treated group at both 1 week (P=0.062) and 6 weeks (P=0.0003) compared to injured-untreated controls (FIG. 4A-4C).

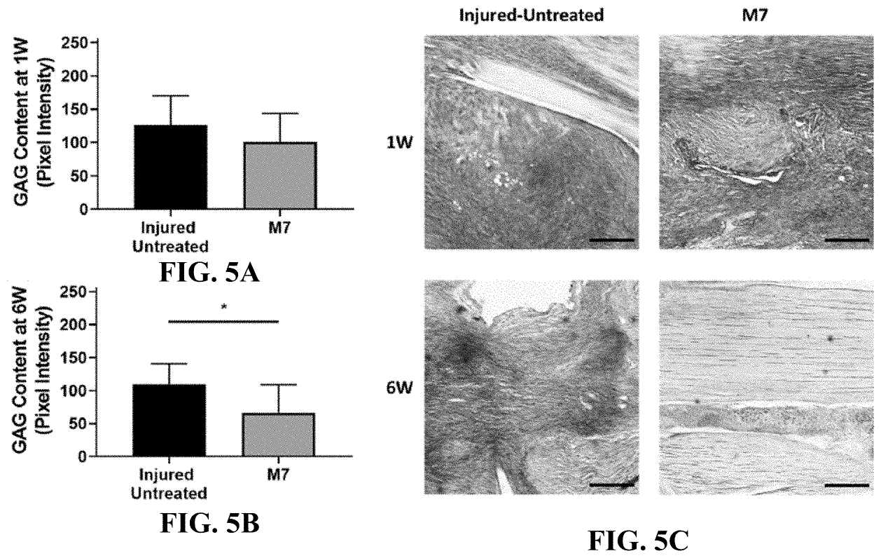

GAG content, as assessed by colorimetric Toluidine blue staining, was lower in M7-treated tendons after 6 weeks compared to injured-untreated controls (P=0.0436). No differences in GAG content between M7-treated tendons and injured-untreated controls was found after 1 week (FIG. 5A-5C).

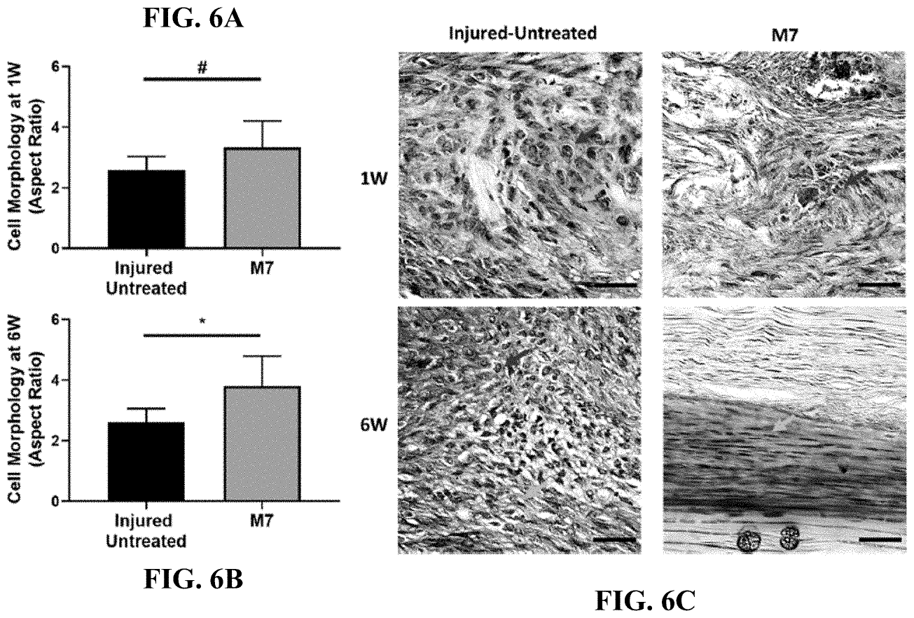

Samples treated with M7 exhibited increased cell aspect ratio compared to injured-untreated controls at both 1 (P=0.0852) and 6 weeks (P=0.0118) post-injury (FIG. 6A-6C).

### Compositional Protein Profile of the Most (M7) and Least (BU) Effective Therapeutics

Proteomic assessment of M7 and BU identified a total of 176 proteins present combined between both groups. A total of 11 proteins differed in concentration between M7 and BU (P<0.1). From these proteins two were glycoproteins, one was an ECM associated protein, and eight were cell processes related proteins. All proteins found to be different between these groups were higher in M7 compared to BU (Table 3). Furthermore, 74 proteins were identified only in M7, two of these were glycoproteins, 12 were ECM associated proteins, and 60 were cell processes related (Table 4).

TABLE 3
Proteomic assessment of most and least effective therapeutic

| | | | | | |
|---|---|---|---|---|---|
| Protein Name | Accession | MW, kDa | BU Abundance | M7 Abundance | Q Value |

Glycoproteins

| | | | | | |
|---|---|---|---|---|---|
| Fibrinogen β | Q3TGR2 | 54.7 | 3.08E+06 ± 2.01E+06 | 1.40E+08 ± 1.74E+08 | 0.0723 |
| chain | | | | | |
| Fibronectin | B9EHT6 | 250.1 | 1.24E+07 ± 9.59E+06 | 2.90E+08 ± 2.72E+08 | 0.0723 |

ECM Associated

| | | | | | |
|---|---|---|---|---|---|
| Myosin-9 | Q8VDD5 | 226.2 | 2.14E+06 ± 2.95E+06 | 7.53E+07 ± 6.07E+07 | 0.0723 |

Cell processes

| | | | | | |
|---|---|---|---|---|---|
| α-Globin 1 | Q91VB8 | 15.1 | 1.56E+06 ± 6.22E+05 | 9.12E+07 ± 9.30E+07 | 0.0723 |
| ATP synthase | P56480 | 56.3 | 2.69E+06 ± 2.18E+06 | 9.43E+07 ± 4.50E+07 | 0.0723 |
| subunit β, | | | | | |
| mitochondrial | | | | | |
| Glyceraldehyde- | A0A0A0MQF6 | 38.6 | 2.14E+06 ± 1.69E+06 | 5.93E+07 ± 3.79E+07 | 0.0723 |
| 3-phosphate | | | | | |
| dehydrogenase | | | | | |
| Histone H2A | Q8BFU2 | 14.1 | 7.80E+06 ± 8.97E+06 | 3.71E+08 ± 1.57E+08 | 0.0723 |
| type 3 | | | | | |
| Histone H3.2 | P84228 | 15.4 | 1.76E+07 ± 2.05E+07 | 3.97E+08 ± 1.86E+08 | 0.0723 |
| Histone H4 | B2RTM0 | 11.4 | 2.39E+07 ± 2.39E+07 | 7.59E+08 ± 4.12E+08 | 0.0723 |
| Tubulin β-5 | P99024 | 49.6 | 2.07E+06 ± 1.86E+06 | 1.91E+08 ± 1.04E+08 | 0.0723 |
| chain | | | | | |
| Vimentin | P20152 | 53.7 | 4.54E+07 ± 5.81E+07 | 8.30E+08 ± 1.88E+08 | 0.0723 |

Note:
A total of 11 proteins were higher in the M7 therapeutic compared to the BU construct.
Abbreviations: BU, B6 uninjured ECM; M7, MRL/MpJ 7-day-provisional-ECM.

TABLE 4
Proteomic assessment of most and least effective therapeutics

| | | |
|---|---|---|
| Accession | Description | MW, kDa |

Glycoproteins

| | | |
|---|---|---|
| Q62009 | Periostin | 93.1 |
| Q61554 | Fibrillin-1 | 312.1 |

ECM Associated Proteins

| | | |
|---|---|---|
| Q3ULT2 | Actinin α4 | 104.9 |
| P10107 | Annexin A1 | 38.7 |
| P48036 | Annexin A5 | 35.7 |
| P14824 | Annexin A6 | 75.8 |
| P11499 | Heat shock protein HSP 90-β | 83.2 |
| F8WJ05 | Inter-α-trypsin inhibitor heavy | 101.6 |
| | chain H1 | |
| Q61704 | Inter-α-trypsin inhibitor heavy | 99.3 |
| | chain H3 | |
| P17742 | Peptidyl-prolyl cis-trans | 18 |
| | isomerase A | |
| P50543 | Protein S100-A11 | 11.1 |
| P27773 | Protein disulfide-isomerase | 56.6 |
| | A3 | |
| Q9Z204 | Heterogeneous nuclear | 34.4 |
| | ribonucleoproteins C1/C2 | |
| P18760 | Cofilin-1 | 18.5 |

Cell Processes

| | | |
|---|---|---|
| Q5M9K7 | 40S ribosomal protein S10 | 18.9 |
| P14131 | 40S ribosomal protein S16 | 16.4 |
| Q5M9L7 | 40S ribosomal protein S17 | 15.5 |
| Q561N5 | 40S ribosomal protein S18 | 17.7 |
| P62267 | 40S ribosomal protein S23 | 15.8 |
| P62855 | 40S ribosomal protein S26 | 13 |
| P97351 | 40S ribosomal protein S3a | 29.9 |
| Q545X8 | 40S ribosomal protein S4 | 29.6 |
| D3YYM6 | 40S ribosomal protein S5 | 20.4 |
| | (Fragment) | |
| Q5BLK1 | 40S ribosomal protein S6 | 28.7 |
| P14206 | 40S ribosomal protein SA | 32.8 |
| P63038 | 60 kDa heat shock protein, | 60.9 |
| | mitochondrial | |
| Q9CXW4 | 60S ribosomal protein L11 | 20.2 |
| P47963 | 60S ribosomal protein L13 | 24.3 |
| E9Q5A0 | 60S ribosomal protein L13a | 48.3 |
| Q9CR57 | 60S ribosomal protein L14 | 23.5 |
| Q58EW0 | 60S ribosomal protein L18 | 21.6 |
| Q9CQM8 | 60S ribosomal protein L21 | 18.6 |
| P62830 | 60S ribosomal protein L23 | 14.9 |
| Q8BP67 | 60S ribosomal protein L24 | 17.8 |
| P14115 | 60S ribosomal protein L27a | 16.6 |
| P27659 | 60S ribosomal protein L3 | 46.1 |
| P62889 | 60S ribosomal protein L30 | 12.8 |
| Q9D1R9 | 60S ribosomal protein L34 | 13.3 |
| P14148 | 60S ribosomal protein L7 | 31.4 |
| P12970 | 60S ribosomal protein L7a | 30 |
| P47738 | Aldehyde dehydrogenase, | 56.5 |
| | mitochondrial | |
| Q8C2Q8 | ATP synthase subunit gamma | 30.2 |
| Q9DB20 | ATP synthase subunit O, | 23.3 |
| | mitochondrial | |
| Q542X7 | Chaperonin subunit 2 (β), | 57.4 |
| | isoform CRA_a | |
| Q8BMK4 | Cytoskeleton-associated | 63.7 |
| | protein 4 | |
| Q99LC5 | Electron transfer flavoprotein | 35 |
| | subunit α, mitochondrial | |
| Q58E64 | Elongation factor 1-α | 50.1 |
| Q3UAD6 | Endoplasmin | 92.4 |
| E9Q561 | Eukaryotic initiation factor | 36.1 |
| | 4A-II | |
| Q8BTM8 | Filamin-A | 281 |
| P68040 | Guanine nucleotide-binding | 35.1 |
| | protein subunit β-2-like 1 | |
| P63017 | Heat shock cognate 71 kDa | 70.8 |
| | protein | |
| P02089 | Hemoglobin subunit β-2 | 15.9 |
| Q9Z2X1 | Heterogeneous nuclear | 45.7 |
| | ribonucleoprotein F | |
| Q8C2Q7 | Heterogeneous nuclear | 51.2 |
| | ribonucleoprotein H | |
| B2RVP5 | Histone H2A | 13.5 |
| P70168 | Importin subunit β-1 | 97.1 |
| Q9D646 | Keratin, type I cuticular Ha4 | 44.5 |
| P08249 | Malate dehydrogenase, | 35.6 |
| | mitochondrial | |
| P35700 | Peroxiredoxin-1 | 22.2 |
| P09411 | Phosphoglycerate kinase 1 | 44.5 |
| P60335 | Poly(rC)-binding protein 1 | 37.5 |
| 054724 | Polymerase I and transcript | 43.9 |
| | release factor | |
| Q922R8 | Protein disulfide-isomerase | 48.1 |
| | A6 | |
| J3QMG5 | Protein Gm5786 | 31.2 |
| D3YWG1 | Protein Gm7964 | 50.9 |
| F6SVV1 | Protein Gm9493 | 21.9 |
| P52480 | Pyruvate kinase PKM | 57.8 |
| A2AVJ7 | Ribosome-binding protein 1 | 158.3 |
| P19324 | Serpin H1 | 46.5 |
| P38647 | Stress-70 protein, | 73.4 |
| | mitochondrial | |
| P68369 | Tubulin α-1A chain | 50.1 |
| Q7TMM9 | Tubulin β-2A chain | 49.9 |
| Q922F4 | Tubulin β-6 chain | 50.1 |

Note:
A total of 74 proteins were identified in M7 that were not present in BU.
Abbreviations: BU, B6 uninjured ECM; M7, MRL/MpJ 7-day-provisional-ECM.

### Discussion

The role of the ECM as a tool to treat pathological conditions has been supported by studies in the heart, kidneys, and esophagus of non-regenerative models.²⁸⁻³⁰ In tendon, however, a hurdle towards the optimization of ECM-derived therapeutics has been the lack of knowledge regarding the effective matrix environment necessary to stimulate improved healing. Interestingly, Applicant has shown that decellularized-ECM constructs from MRL/MpJ provide the necessary biochemical cues to modulate canonically healing B6 cell behavior in vitro towards the re-generative phenotype of MRL/MpJ cells during in vivo healing.¹⁶ Therefore, in this Example Applicant harnessed the compositional properties of the MRL/MpJ healing environment and assessed the ability of its ECM to improve scar-mediated tendon healing in vivo.

Following injury, the compositional properties of the tendon ECM continuously remodel over time, providing different cues that guide cell behavior throughout the stages of healing.^{17,18} Therefore, the unique biological contributions of ECMs harnessed from different early healing timepoints give rise to distinct therapeutic potentials depending on the time of ECM acquisition. Supporting this notion, Applicant evaluated the therapeutic capabilities of ECM from tendons after 3 and 7-days post-injury and identified that MRL/MpJ-ECM after 7-days harnesses the potential to improve structural, mechanical, compositional, and cellular behavior of scar-mediated healing tendons.

The loss of structural and mechanical integrity resulting from the ineffective canonical tendon healing response, leads to high incidences of re-injury and tendon rupture during physical activity.²⁻⁴

Interestingly, the analysis showed that M7 treatment possesses the ability to improve the tendon healing response by stimulating the deposition of matrix with enhanced structural qualities. Still, the main physiological role of tendons is to transfer high levels of loading from muscles to bones during locomotion.^{18,19,31} Therefore, while matrix alignment is a key structural characteristic of healthy tissues, improvements in this metric, without functional assessment, are not sufficient to completely determine therapeutic efficiency.

Analysis of tissue stiffness showed that M7 treatment was cap-able of improving the functional outcome of the healing tendons. The increased stiffness following treatment could result in deviations to the mechanical environment experienced by cells compared to the injured-untreated environment throughout healing.′ In turn, it is possible that cells respond to these changes in mechanical stimulus by depositing higher quality matrix that allows tendons to more effectively resist deformation during sub-rupture loading. Interestingly, mechanical analysis revealed a lack of improvement in ultimate load regardless of treatment. While surprising, this lack of improvement could be due to the ineffective integration of newly deposited matrix at the injury site with the native tendon core, thereby resulting in the formation of stress concentrations at this interface that are susceptible to failure during high levels of loading. Additionally, no changes were identified in the stress relaxation of the treatment groups

compared to injured-untreated, or uninjured controls. This finding was not surprising, as previous studies assessing the mechanical properties of canonically healing tendons have identified that structural and compositional changes following injury do not always translate to changes in the bulk tissue stress relaxation. For instance, others have identified that introduction of an incisional injury to patellar or flexor digitorum longus tendons did not result in changes to the stress relaxation parameter after three weeks of healing compared to uninjured controls.33 Furthermore, studies utilizing genetically modified mice showed that the absence of biglycan, a proteoglycan that modulates tissue stiffness and ultimate load, does not lead to changes in stress relaxation compared to a wildtype B6.34 However, while changes in the stress relaxation of the treated ten-dons are not being manifested at the tissue level, future work assessing cell level mechanics or changes in fiber sliding throughout the relaxation period could further elucidate the effect of treatment with the ECM-derived constructs on the tendon viscoelastic properties. Nevertheless, its ability to modulate the tendon healing response towards both improved structure and function, labeled M7 as the most effective therapeutic.

To further interrogate the hypothesis and elucidate the compositional differences that lead to improved versus ineffective healing, Applicant performed proteomic assessment of the M7 construct and compared it to the least effective BU therapeutic. Glycoproteins such as fibronectin and fibrinogen were higher in M7 compared to BU. These proteins are an essential stimulus for cell migration, adhesion and integrin receptor interactions.³⁵⁻³⁷ The findings showed that treatment with the glycoprotein-rich environment of M7, led to early and late decreases in collagen III content and increases in cell elongation from untreated canonically healing tendons. Thereby, early introduction of these com-ponents via M7 treatment can result in changes to the mechanotransduction environment that could entail a possible mechanism for the observed improvements in cell morphology and matrix deposition. While these findings suggest a beneficial role for the exogenous addition of these glycoproteins during early healing, retention of a high GAG content into the remodeling phase has been identified as a marker of the scar-mediated healing response of canonically healing tendons.^{19,38}

On the other hand, studies have shown that a hallmark of the scarless-healing MRL/MpJ model is a decrease in GAG content compared to B6 between 4- and 8-weeks post-injury.14 Similarly, Applicant identified a decrease in GAG content by the 6-week timepoint following M7 treatment compared to injured-untreated controls, elucidating signs of beneficial matrix remodeling and further highlighting the potential of this

therapeutic to encourage scarless behavior in a non-regenerative model. Moreover, the M7 constructs contained 74 proteins that are not

present in BU. Interestingly, out of these proteins, Fibrillin-1 is as-sociated with the regulation of insulin-like growth factor (IGF) transport and uptake.39 Studies have shown that IGF signaling is necessary for proper tendon growth and maturation in adults, thereby highlighting a promising role for this glycoprotein in mod-ulating the growth factor environment necessary for the improved healing response following M7 treatment.39,40 Additionally, heat shock protein 90, was identified in M7 but not BU. Interestingly, studies have elucidated this anti-apoptotic protein as a promising

target for tendon therapeutics, as it has been implicated in a natural attempt for tendons to protect cells from increased oxidative stresses and prevent programmed cell death following injury.41 It is possible that a transient induction of these proteins through the therapeutic, beneficially modulates the apoptotic response of cells by keeping tenocytes alive to encourage tissue remodeling and effective matrix deposition at the injury site. However, due to the limited number of studies that have interrogated the role of HSP 90 in tendons, future work is necessary to assess the specific role of this protein during scarless healing of acute tendinopathies.

Lastly, while the true function of proteins found in M7 and not BU such as 40S ribosomal protein S6, pyruvate kinase PKM, guanine nucleotide-binding protein subunit β-2-like 1 and 60S ribosomal proteins remains unknown, these have been implicated in cell processes such as regulation of mRNA translation, metabolism, growth, apoptosis and proliferation.42-44 Therefore, while identification of this protein profile is a necessary first step to identify the compositional players involved in the outcomes observed in this study, future work is necessary to understand the specific roles of these proteins in tendon healing.

This study is not without limitations. Despite the improvements of M7-treated tendons compared to injured-untreated controls, this ECM-derived therapeutic did not restore treated tendon's structure and mechanics to pre-injury levels. To overcome this limitation, undergoing studies are assessing the role of specific proteins within the MRL/MpJ ECM after 7 days post-injury (M7) at modulating B6 cell behavior towards a scarless phenotype. This new analysis, coupled with an optimized delivery system for individual proteins, could allow us to narrow down the M7 components that could be isolated and translated into more effective protein-based therapeutics that may be beneficial in future clinical applications. Moreover, while this work has identified a beneficial role for therapeutics derived from MRL/MpJ ECM after 7 days of healing, future work elucidating the therapeutic potential of MRL/MpJ ECM harnessed from later timepoints throughout the healing response could help further interrogate the specific compositional profile that leads to improved canonical tendon healing.

Surprisingly, mechanical assessment of the constructs showed that introduction of PEG-4MAL alone resulted in a decrease in stiffness of treated tendons compared to injured-untreated controls. Recently, this delivery mechanism has been effectively utilized as a mechanical pillow to ameliorate damage in a post-traumatic osteoarthritis model.²¹ However, the results indicate that while this hydrogel may be useful for intra-articular applications, its compatibility with the tendon healing environment may be limited. The mechanical forces experienced by the gel when placed inside the joint are largely compressive compared to the tensile nature of the tendon environment. Therefore, it is likely that the differences in the biomechanical stimulus experienced in the joint cavity compared to tendons, is responsible for the lack of effectiveness of this delivery mechanism as applied to this study. Nevertheless, despite the use of a delivery system that caused further deviations from uninjured controls, treatment with M7 resulted in improved matrix structure, composition and function compared to injured-untreated tendons. Therefore, future work identifying an improved delivery system that is more compatible with the tensile nature of the tendon healing environment, could help further improve the healing response of tendons treated with M7 closer to pre-injury levels.

Furthermore, Applicant has previously shown that both males and females exhibit improved healing compared to B6 in regard to their mechanical outcome.⁴⁵ Therefore, as a first step to interrogate the therapeutic potential of MRL/MpJ derived constructs Applicant utilized male mice as the focus of this work. However, studies have identified differences in the quantitative trait loci that influence the MRL/MpJ healing response between male and females.⁴⁶ These genetic differences could manifest in the composition of ECM deposited at the injury site between sexes. Therefore, future work is necessary to identify whether ECM therapeutics derived from female mice exhibit similar potential at improving the tendon healing response. Lastly, tendon area was not calculated in this study. This metric is usually obtained from digital images acquired during mechanical testing, however, when submerged into the water bath, loose connective tissue protruding from the tendon at the sites of dissection limited

the ability of accurate cross-sectional area measurements through visual interpretation. Therefore, to preserve the accuracy of the results Applicant did not measure material properties such as Young's modulus and ultimate stress. Laser-based methods, ultrasound imaging or use of a profilometer could be applied in future studies to increase the accuracy these measurements.^{47,48}

Overall, Applicant has at least shown the ability of MRL/MpJ ECM-derived constructs to improve the structural, functional and cellular behavior of canonically healing tendons. Additionally, this work elucidates that incorporation of a glycoprotein rich therapeutic during early healing provides a valuable platform for tendon regeneration and highlights promising targets such as Fibrillin-1 and HSP 90 for the development of future tendon therapeutics and tissue engineering applications.

### REFERENCES FOR EXAMPLE 1

1. Screen H R C, Berk D E, Kadler K E, et al. Tendon functional extra-cellular matrix. J Orthop Res. 2015; 33(6):793-799. https://www.ncbi.nlm.nih.gov/pubmed/25640030
2. Lin T W, Cardenas L, Soslowsky L J. Biomechanics of tendon injury and repair. J Biomech. 2004; 37(6):865-877. http://www.sciencedirect.com/science/article/pii/S0021929003004068
3. Nourissat G, Berenbaum F, Duprez D. Tendon injury: from biology to tendon repair. Nat Rev Rheumatol. 2015; 11(4): 223-233. https://doi org/10.1038/nrrheum.2015.26
4. Andarawis-Puri N, Flatow E L. Promoting effective tendon healing and remodeling. J Orthop Res. 2018; 36(12):3115-3124. https://www.ncbi.nlm.nih.gov/pubmed/30175859
5. Zhang J, Pan T, Liu Y, Wang J H-C. Mouse treadmill running enhances tendons by expanding the pool of tendon stem cells (TSCs) and TSC-related cellular production of collagen. J Orthop Res. 2010; 28(9): 1178-1183. https://doi.org/10.1002/jor.21123
6. Paredes J J, Andarawis-Puri N. Therapeutics for tendon regeneration: a multidisciplinary review of tendon research for improved healing. Ann NY Acad Sci. 2016; 1383(1): 125-138. https://www.ncbi.nlm.nih.gov/pubmed/27768813
7. Thomopoulos S, Kim H M, Das R, et al. The effects of exogenous basic fibroblast growth factor on intrasynovial flexor tendon healing in a canine model. J Bone Joint Surg Am. 2010; 92(13):2285-2293.
8. Yang G, Rothrauff B B, Tuan R S. Tendon and ligament regeneration and repair: clinical relevance and developmental paradigm. Birth De-fects Res C Embryo Today. 2013; 99(3):203-222.
9. Hitchcock T F, Light T R, Bunch W H, et al. The effect of immediate constrained digital motion on the strength of flexor tendon repairs in chickens. J Hand Surg Am. 1987; 12(4):590-595. https://doi.org/10.1016/S0363-5023(87)80213-7
10. Rijal G. The decellularized extracellular matrix in regenerative medicine. Regen Med. 2017; 12(5):475-477. https://doi.org/10.2217/rme-2017-0046
11. Urciuolo A, Urbani L, Perin S, et al. Decellularised skeletal muscles allow functional muscle regeneration by promoting host cell migration. Sci Rep. 2018; 8(1):8398. https://doi.org/10.1038/s41598-018-26371-y
12. Chen W C W, Wang Z, Missinato M A, et al. Decellularized zebrafish cardiac extracellular matrix induces mammalian heart regeneration. Sci Adv. 2016; 2(11):e1600844.
13. Paredes J, Shiovitz D A, Andarawis-Puri N. Uncorrelated healing response of tendon and ear injuries in MRL highlight a role for the local tendon environment in driving scarless healing. Connect Tissue Res. 2018; 59(5):472-482.
14. Sereysky J B, Flatow E L, Andarawis-Puri N. Musculoskeletal re-generation and its implications for the treatment of tendinopathy. Int J Exp Pathol. 2013; 94(4):293-303.
15. Lalley A L, Dyment N A, Kazemi N, et al. Improved biomechanical and biological outcomes in the MRL/MpJ murine strain following a full-length patellar tendon injury. J Orthop Res. 2015; 33(11):1693-1703.
16. Paredes J, Marvin C J, Vaughn B, Andarawis-Puri N. Innate tissue properties drive improved tendon healing in MRL/MpJ and harnesses cues that enhance behavior of canonical healing cells. FASEB. 2020; 1-16.
17. Molloy T, Wang Y, Murrell G. The roles of growth factors in tendon and ligament healing. Sports Med. 2003; 33(5):381-394.
18. Thomopoulos S, Parks W C, Rifkin D B, Derwin K A. Mechanisms of tendon injury and repair. J Orthop Res. 2015; 33(6):832-839.
19. Oakes B W. Tissue healing and repair: tendons and ligaments. Rehab Sports Injuries: Scientific Basis. 2003:28-98.
20. Gillies A R, Smith L R, Lieber R L, Varghese S. Method for decellularizing skeletal muscle without detergents or proteolytic enzymes. Tissue Eng Part C Methods. 2011; 17(4):383-389.
21. Holyoak D T, Wheeler T A, van der Meulen M C H, Singh A. Injectable mechanical pillows for attenuation of load-induced post-traumatic osteoarthritis. Regen Biomater. 2019; 6(4):211-219.
22. Lee T T, Garcia J R, Paez J I, et al. Light-triggered in vivo activation of adhesive peptides regulates cell adhesion, inflammation and vascularization of biomaterials. Nat Mater. 2015; 14(3):352-360.
23. Purwada A, Shah S B, Beguelin W, et al. Ex vivo synthetic immune tissues with T cell signals for differentiating antigen-specific, high affinity germinal center B cells. Biomaterials. 2019; 198:27-36.
24. Bell R, Boniello M R, Gendron N R, et al. Delayed exercise promotes remodeling in sub-rupture fatigue damaged tendons. J Orthop Res. 2015; 33(6):919-925.
25. Yang Y, Thannhauser T W, Li L, Zhang S. Development of an integrated approach for evaluation of 2-D gel image analysis: impact of multiple proteins in single spots on comparative proteomics in conventional 2-D gel/MALDI workflow. Electrophoresis. 2007; 28(12):2080-2094.
26. Thomas C J, Cleland T P, Zhang S, et al. Identification and characterization of glycation adducts on osteocalcin. Anal Biochem. 2017; 525: 46-53.
27. Yang Y, Anderson E, Zhang S. Evaluation of six sample preparation procedures for qualitative and quantitative proteomics analysis of milk fat globule membrane. Electrophoresis. 2018; 39(18):2332-2339.
28. Figliuzzi M, Bonandrini B, Remuzzi A. Decellularized kidney matrix as functional material for whole organ tissue engineering. J Appl Biomater Funct Mater. 2017; 15(4):e326-e333.
29. Kc P, Hong Y, Zhang G. Cardiac tissue-derived extracellular matrix scaffolds for myocardial repair: advantages and challenges. Regen Biomater. 2019; 6(4):185-199.
30. Riganti J M, Ciotola F, Amenabar A, et al. Urinary bladder matrix scaffolds strengthen esophageal hiatus repair. JSurgRes. 2016; 204(2):344-350.
31. Stauber T, Blache U, Snedeker J G. Tendon tissue microdamage and the limits of intrinsic repair. Matrix Biol. 2019; 85-86:68-79.
32. Subramanian A, Kanzaki L F, Galloway J L, Schilling T F. Mechanical force regulates tendon extracellular matrix organization and tenocyte morphogenesis through TGFbeta signaling. eLife. 2018:7.
33. Beason D P, Kuntz A F, Hsu J E, et al. Development and evaluation of multiple tendon injury models in the mouse. J Biomech. 2012; 45(8): 1550-1553.
34. Dourte L M, Pathmanathan L, Mienaltowski M J, et al. Mechanical, compositional, and structural properties of the mouse patellar tendon with changes in biglycan gene expression. J Orthop Res. 2013; 31(9): 1430-1437.
35. Dejana E, Lampugnani M G, Giorgi M, et al. Fibrinogen induces endothelial cell adhesion and spreading via the release of endogenous matrix proteins and the recruitment of more than one integrin re-ceptor. Blood. 1990; 75(7):1509-1517.
36. Johansson S, Svineng G, Wennerberg K, et al. Fibronectin-integrin interactions. Front Biosci. 1997; 2:d126-d146.
37. Hsiao C-T, Cheng H-W, Huang C-M, et al. Fibronectin in cell adhesion and migration via N-glycosylation. Oncotarget. 2017; 8(41): 70653-70668.
38. Attia M, Scott A, Duchesnay A, et al. Alterations of overused supraspinatus tendon: a possible role of glycosaminoglycans and HARP/pleiotrophin in early tendon pathology. J Orthop Res. 2012; 30(1):61-71.
39. Disser N P, Sugg K B, Talarek J R, et al. Insulin-like growth factor 1 signaling in tenocytes is required for adult tendon growth. FASEB J. 2019; 33(11):12680-12695.
40. Provenzano P P, Alejandro-Osorio A L, Grorud K W, et al. Systemic administration of IGF-I enhances healing in collagenous extracellular matrices: evaluation of loaded and unloaded ligaments. BMC Physiol. 2007; 7:2.
41. Millar N L, Wei A Q, Molloy T J, et al. Heat shock protein and apoptosis in supraspinatus tendinopathy. Clin Orthop Relat Res. 2008; 466(7): 1569-1576.
42. Robledo S, Idol R A, Crimmins D L, et al. The role of human ribosomal proteins in the maturation of rRNA and ribosome production. RNA. 2008; 14(9):1918-1929.
43. Du Y, Meng J, Huang Y, et al. Guanine nucleotide-binding protein subunit beta-2-like 1, a new Annexin A7 interacting protein. Biochem Biophys Res Commun. 2014; 445(1):58-63.
44. Desai S, Ding M, Wang B, et al. Tissue-specific isoform switch and DNA hypomethylation of the pyruvate kinase PKM gene in human cancers. Oncotarget. 2014; 5(18):8202-8210.
45. George N, Bell R, Paredes J, et al. Superior mechanical recovery in male and female MRL/MpJ tendons is associated with a unique genetic profile. JOrthopRes. 2020. https://doi.org/10.1002/jor.24705
46. Heber-Katz E, Chen P, Dvm L C, et al. Regeneration in MRL mice: further genetic loci controlling the ear hole closure trait using MRL and M.m. Castaneus mice. Wound Repair Regen. 2004; 12(3):384-392.
47. Pokhai G G, Oliver M L, Gordon K D. A new laser reflectance system capable of measuring changing cross-sectional area of soft tissues during tensile testing. J Biomech Eng. 2009; 131(9): 94504.
48. Moon D K, Abramowitch S D, Woo S L-Y. The development and vali-dation of a charge-coupled device laser reflectance system to measure the complex cross-sectional shape and area of soft tissues. J Biomech. 2006; 39(16):3071-3075.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition for treating tendon injury, the composition comprising:
a decellularized tendon extracellular matrix (ECM) composition comprising one or more bioactive factors having a tendon injury induced bioactive factor profile.

2. The composition of claim 1, wherein the decellularized tendon ECM composition comprises pulverized decellularized tendon ECM.

3. The composition of claim 1, wherein the decellularized tendon ECM composition comprises a hydrogel.

4. The composition of claim 3, wherein the hydrogel is PEG-4MAL.

5. The composition of claim 1, wherein the tendon injury induced bioactive factor profile is a 7-day post injury tendon injury induced bioactive factor profile.

6. The composition of claim 1, wherein the tendon injury induced bioactive factor profile comprises one or more proteins selected from Fibrinogen β chain, fibronectin, Myosin-9, α-Globin 1, ATP synthase subunit β, mitochondrial, Glyceraldehyde-3-phosphate dehydrogenase, Histone H2A type 3, Histone H3.2, Histone H4, Tubulin β-5 chain, Vimentin, periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein S100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof.

7. The composition of claim 1, wherein one or more of the bioactive factors in the one or more tendon injury induced bioactive factor profile are recombinant proteins.

8. The composition of claim 1, wherein none of the bioactive factors in the one or more tendon injury induced bioactive factor profile are recombinant proteins.

9. The composition of claim 1, wherein the decellularized tendon composition is prepared from a tendon harvested from a donor about 7 days post tendon injury.

10. A composition for treating tendon injury, the composition comprising:
a protein composition comprising a plurality of bioactive factors, wherein the protein composition comprises a tendon injury induced bioactive factor profile.

11. The composition of claim 10, wherein the tendon injury induced bioactive factor profile is a 7-day post injury tendon injury induced bioactive factor profile.

12. The composition of claim 10, wherein the tendon injury induced bioactive factor profile comprises one or more proteins selected from Fibrinogen β chain, fibronectin, Myosin-9, α-Globin 1, ATP synthase subunit β, mitochondrial, Glyceraldehyde-3-phosphate dehydrogenase, Histone H2A type 3, Histone H3.2, Histone H4, Tubulin β-5 chain, Vimentin, periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein 5100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof.

13. The composition of claim 10, wherein one or more of the plurality of bioactive factors are isolated from decellularized tendon extracellular matrix (ECM).

14. The composition of claim 13, wherein decellularized tendon ECM is post-injury decellularized tendon ECM, optionally day 7 post-injury decellularized tendon ECM.

15. The composition of claim 10, wherein one or more of the plurality of bioactive factors are recombinant proteins.

16. The composition of claim 10, wherein the composition further comprises a hydrogel.

17. The composition of claim 16, wherein the hydrogel is PEG-4MAL.

18. A method of treating a tendon injury, the method comprising:
administering a composition as in claim 1 to a subject in need thereof, optionally at the site of a tendon injury.

19. The method of claim 18, wherein the composition is effective to increase tendon stiffness, improve tendon matrix alignment, or both.

20. The method of claim 18, wherein the tendon injury induced bioactive factor profile is a 7-day post injury tendon injury induced bioactive factor profile, and optionally wherein the a 7-day post injury tendon injury induced bioactive factor profile comprises one or more proteins selected from Fibrinogen β chain, fibronectin, Myosin-9, α-Globin 1, ATP synthase subunit β, mitochondrial, Glyceraldehyde-3-phosphate dehydrogenase, Histone H2A type 3, Histone H3.2, Histone H4, Tubulin β-5 chain, Vimentin, periostin, fibrillin-1, Actinin α4, Annexin A1, Annexin A5, Annexin A6, Heat shock protein HSP 90-β, Inter-α-trypsin inhibitor heavy chain H1, Inter-α-trypsin inhibitor heavy chain H3, Peptidyl-prolyl cis-trans isomerase A, Protein S100-A11, Protein disulfide-isomerase A3, Heterogeneous nuclear ribonucleoproteins C1/C2, Cofilin-1, 40S ribosomal protein S10, 40S ribosomal protein S16, 40S ribosomal protein S17, 40S ribosomal protein S18, 40S ribosomal protein S23, 40S ribosomal protein S26, 40S ribosomal protein S3a, 40S ribosomal protein S4, 40S ribosomal protein S5 or fragment thereof, 40S ribosomal protein S6, 40S ribosomal protein SA, mitochondrial 60 kDa heat shock protein, 60S ribosomal protein L11, 60S ribosomal protein L13, 60S ribosomal protein L13a, 60S ribosomal protein L14, 60S ribosomal protein L18, 60S ribosomal protein L21, 60S ribosomal protein L23, 60S ribosomal protein L24, 60S ribosomal protein L27a, 60S ribosomal protein L3, 60S ribosomal protein L30, 60S ribosomal protein L34, 60S ribosomal protein L7, 60S ribosomal protein L7a, mitochondrial Aldehyde dehydrogenase, ATP synthase subunit gamma, mitochondrial ATP synthase subunit O, Chaperonin subunit 2 (β) isoform CRA_a, Cytoskeleton-associated protein 4, mitochondrial Electron transfer flavoprotein subunit α, Elongation factor 1-α, Endoplasmin, Eukaryotic initiation factor 4A-II, filamin-A, Guanine nucleotide binding protein subunit β-2-like 1, Heat shock cognate 71 kDa protein, Hemoglobin subunit β-2, Heterogeneous nuclear ribonucleoprotein F, Heterogeneous nuclear ribonucleoprotein H, Histone H2A, Importin subunit β-1, Keratin-type I cuticular Ha4, mitochondrial malate dehydrogenase, Peroxiredoxin-1, Phosphoglycerate kinase 1, Poly(rC)-binding protein 1, Polymerase I and transcript release factor, Protein disulfide-isomerase A6, Protein Gm5786, Protein Gm7964, Protein Gm9493, Pyruvate kinase PKM, Ribosome-binding protein 1, Serpin H1, Stress-70 protein, mitochondrial, Tubulin α-1A chain, Tubulin β-2A chain, Tubulin β-6 chain, and any combination thereof.
